(19) United States
(12) Patent Application Publication
(10) Pub. No.: US 2025/0093323 A1
(43) Pub. Date: Mar. 20, 2025

(54) TENSIOMETER AND METHOD FOR DETERMINING A SPATIALLY AVERAGED WATER POTENTIAL IN A TEST PIECE

(71) Applicant: Membran Tech GmbH, Cottbus (DE)

(72) Inventors: Detlef Lazik, Salzatal (DE); Gerrit De Rooij, Halle (DE)

(73) Assignee: Membran Tech GmbH, Cottbus (DE)

(21) Appl. No.: 18/730,437

(22) PCT Filed: Jan. 20, 2023
(86) PCT No.: PCT/EP2023/051365

§ 371 Date: Jul. 19, 2024

(30) Foreign Application Priority Data
Jan. 21, 2022 (DE) 10 2022 200 685.2

(51) Int. Cl. G01N 33/24; G01N 13/02; G01N 13/04

(52) U.S. Cl. CPC G01N 33/246; G01N 13/02; G01N 13/04

(57) ABSTRACT

A tensiometer comprising a measuring cell of a selectable length, referred to as a potential cell, which comprises a closable measuring chamber sheathed by a tubular semipermeable membrane, fittable into a test piece; a reference system comprising a reference chamber and a measuring cell of a selectable length, referred to as a reference cell and comprising a closable measuring chamber sheathed by a tubular semipermeable membrane, the reference cell being integrated into the reference chamber of the reference system, and wherein the reference chamber contains an osmotic reference solution or can be filled with such a solution, wherein the potential cell and the reference cell are filled or can be filled with an osmotic solution; and a pressure-measuring device, that measures at least a pressure difference (Δp) between the cell measuring chambers. The invention also relates to a method.

### BACKGROUND OF THE INVENTION

### 1. Field of the Invention

The invention relates to a tensiometer and a method for determining a spatially averaged water potential in a test piece.

### 2. Brief Description of the Related Art

### Background

Plants need to overcome the water potential of soil in order to extract water from it. This water potential consists of the gravimetric potential, the matrix potential and the osmotic potential. In humid unsaturated conditions, the locally provided water potential is generally equal to the matrix potential—the osmotic potential can generally be ignored. In arid soils, the concentration of dissolved salts can lead to an increased contribution of the osmotic potential to the water potential, in particular due to the evaporation of the water in the soil near the surface, which can no longer be ignored. External conditions such as precipitation or irrigation, air humidity, temperature, evaporation and locally varying internal swelling/sinking phenomena in the soil, such as gravity-induced water extraction, water extraction via the plant roots or the binding of water to hygroscopic substances (often salts) constantly cause localized changes in the water potential, which in turn cause water flows that vary over time and space.

### Prior Art

The water potential is measured by tensiometers which are either directly and indirectly measuring tensiometers according to the measuring principle. Direct measurement is based on a water-filled cavity encapsulated by a porous filter cartridge in capillary contact with the soil and provided with a pressure sensor. If the external water potential causes water to be extracted from the cavity, this leads to a drop in pressure in this type of tensiometer. The resulting pressure difference from atmospheric pressure (also referred to as suction tension) corresponds at equilibrium to the water or matrix potential. In indirect methods, the water potential in the soil is made equal to a separate hygroscopic medium in which the water activity or the water potential can be measured in a suitable manner. Measurement methods are based for example on the change in the capacitance of a capacitor dielectric consisting of the medium, the electrical conductivity of the medium, the propagation of heat pulses through the medium or also the pressure in this medium.

Particularly in dry conditions, i.e. where the demand for irrigation water reaches the limits of the water available under economic conditions, irrigation strategies are required that reliably supply plants with water before they reach a permanent wilting point (plants are irreparably damaged). Within the range of the permanent wilting point, the water potentials can reach −1.5 MPa to −2.5 MPa in extreme cases. The roots now suction, figuratively, with a hanging water column of height Δh=ρ_{w}gΨ_{w} (water density ρ_{w}, gravitational constant g, water potential Ψ_{w}) of 15000 . . . 25000 cmWs (cm water column) equivalent to the negative pressure on the remaining water still physically bound in capillaries and on surfaces (water films, gusset fillings). In directly measuring tensiometers, the water would boil in these conditions, so they can no longer be used in these ranges. Even at a suction voltage of 700 to 800 cmWs, water vapor bubbles form in this type of tensiometer, making it impossible to measure the external water potential. Indirectly measuring tensiometers, for example the polymer tensiometer, show their strengths in this range. The measuring chamber of this type of tensiometer fitted with a pressure sensor is filled with a hygroscopic polymer that couples to the external water via a microporous membrane, for example microporous aluminum oxide, through a porous cartidge that is pressure-stable up to 15 bar for example. The hygroscopic polymers mentioned in the literature are substances such as polyethylene glycol (PEG), polyacrylamide (Praestol 2500) or polysaccharide (Dextran 500), which cannot penetrate the microporous membrane due to their molecular size, but on the other hand bind incoming water and reduce its activity, so that further water is removed from the environment. This creates an overpressure (osmotic pressure) compared to the pressure of the water in the vicinity of the tensiometer. In the equilibrium of the chemical potentials of the water in the polymer, determined by its osmotic potential and in the soil, determined by its water potential, this overpressure shifts in proportion to the external water potential. If the external water potential drops, the soil draws water from the polymer tensiometer and the fluid pressure in the polymer drops and vice versa. As long as the pressure in the polymer tensiometer is greater than the water vapor pressure, the formation of water vapor bubbles is prevented. This pressure, which limits the measurement range, is determined by the type of polymer and approximately coincides with the osmotic potential of the polymer that is oversaturated with water.

Problems with the Polymer Tensiometer

After calibrating the polymer tensiometer against a freely moving water phase at atmospheric pressure (referred to in the following as standard conditions) and taking temperature dependencies into account, the water potential of the soil can be determined. In principle, this tensiometer principle is particularly suitable for deep (large) water potentials and large measurement ranges, as the reference point of the measuring arrangement is determined by the osmotic potential of the polymer. If this is compensated for approximately by the water potential in the test piece, the pressure in the tensiometer approaches the zero point (close to the water vapor pressure).

The time required to reach equilibrium limits the volume of the measuring chamber filled with the polymer. For this reason, polymer tensiometers with a significantly minimized measuring chamber volume at maximized exchange surface have been developed, for example with measuring chambers with a chamber height of 1.1 to 2.5 mm and a radius of 8.45 mm and conical exchange surfaces in the range of 167 . . . 260 mm². What can be regarded as an important optimization step, with regard to minimizing the setting time, requires the local reference of the measurement from the point of view of the field application.

With regard to an efficient control of an irrigation system for example, a water potential sensor that averages the locally different water potentials would be desirable. In particular, it would be desirable to be able to measure this average water potential over a lateral range, the size of which would be sufficient for example to allow representative averaging over soil areas with different levels of root penetration. Such a spatial averaging range is characterized by the soil-dependent dimension of the so-called pedon, a soil section with a volume that is sufficiently large that the relevant properties of the soil are statistically averaged (representative) depending on the location. This pedon is often regarded as a section of soil with a lateral extension of around 1 m, which is often used as the basis for the diameter of lysimeters. Depending on the soil type, but also on its plant stock the actual averaging length (pedon) required for the representative determination of the water potential will be in particular in a range from 1 dm to more than 10 m.

### SUMMARY OF THE INVENTION

### Objective

The objective of the invention is to create a robust, indirectly measuring tensiometer and a method that enables a representative determination of water potential or matrix potential in a heterogeneously composed test piece, such as a pedonscale section of a soil body. The tensiometer and the method based thereon should make it possible, for both moist (humid) and dry soils (arid climate regions), to determine the locally varying water potentials in a representative averaged manner. Furthermore, in order to optimally adapt the measuring accuracy and the measurement range to the respective application, in particular: i) the reference point of the measuring arrangement should be moved to the water potential of the free water surface at atmospheric pressure (standard conditions), since the greatest accuracy has be achieved here naturally, ii) further reference potentials should also be adjustable depending on the problem and iii) the width of the measurement range should be adjustable by the user depending on the problem or should be presentable depending on the use.

### Implementation According to the Invention

The indirectly measuring tensiometer introduced in the following for measuring water potential or matrix potential is provided with a reference system and is based on at least two non-porous, semipermeable membrane tubes made of polymers that are suitable in some cases, which are filled with osmotic solutions and sealed. Due to the nature of the membranes, substances can only pass through them diffusively. A pressure difference between the osmotic solutions is measured using suitable pressure sensors. Furthermore, a method is introduced according to which the pressure difference detected by means of the tensiometer (according to one of the embodiments described in this disclosure) provides a measure averaged over the length of the membrane tube that corresponds to the spatially averaged matrix potential of the water in the test piece if the reference potential and membrane material are suitably selected. Tensiometers and methods thus make it possible, as explained below, to spatially average the water potential or the matrix potential in the test piece, for example a pedonscale soil body.

In particular, a tensiometer is created, comprising a measuring cell of selectable, referred to as a potential cell, which comprises a closable measuring chamber sheathed in a tubular semipermeable membrane and can be installed in a test piece; a reference system comprising a reference chamber and a measuring cell of selectable length, referred to as a reference cell, which comprises a closable measuring chamber sheathed in a tubular semipermeable membrane, wherein the reference cell is integrated into the reference chamber of the reference system, and wherein the reference chamber contains an osmotic reference solution or can be filled with such a solution, wherein the potential cell and the reference cell are filled or can be filled with an osmotic solution; and a pressure measuring device, which is set up to measure at least one pressure difference between the measuring chambers of the potential cell and the reference cell.

Furthermore, in particular a method for determining a spatially averaged water potential in a test piece is provided, wherein at least the pressure difference between the measuring chambers of the potential cell and the reference cell is detected by means of the tensiometer according to any one of the described embodiments, which due to its arrangement inside the reference chamber of the reference system, permits reference to a selectable reference potential, wherein on the basis of the detected pressure difference as a function of the semipermeable membrane used and the reference potential the water and/or matrix potential in the test piece, averaged over the length of the potential cell, is determined and is provided as a measurement value.

### Components of the Tensiometer and Terms Used

The tensiometer comprises a potential cell and a reference system, and also in particular a tensiometer board and suitable housing components according to the design (the reference signs are given with reference to the Figures).

The potential cell comprises a membrane tube, the outer surface of which is in contact with a substrate in the test piece for measuring the water potential and the inner surface of which delimits a measuring chamber, in particular radially.

The reference system comprises a reference chamber filled with a reference solution, into which a reference cell is integrated.

The reference cell comprises a membrane tube, the outer surface of which is in contact with the reference solution for measuring a reference potential and the inner surface of which delimits a further measuring chamber, in particular radially.

The measuring chambers are formed in particular by the respective membrane tubes.

The potential cell and reference cell are configured in particular as radially symmetrical measuring cells, which can have suitable measuring devices and functional elements (for volume reduction and mechanical stabilization). The measuring chambers of both measuring cells which can be closed with valves in particular are filled with the same osmotic solutions.

The tensiometer board contains an integrated electronic circuit for data detection, processing, evaluation, storage and/or output as well as suitable connection options depending on the design.

The tensiometer is powered by an internal or external voltage source.

### Functional Principle

The functional principle of the tensiometer is explained in the following in more detail, based in particular on identical measuring cells. The potential cell should be exposed externally to a test piece, in particular the soil. The reference cell should be located in the reference chamber filled with the reference substance, e.g. water, at air pressure (standard conditions). Both measuring cells are locally exposed to the same external temperature and are sealed off from one another and from the outside and initially contain the same concentrations of a hygroscopic substance dissolved in water. A pressure measuring device records the pressure difference Δp between the measuring chambers of the potential cell and reference cell. Alternatively, for this purpose, the pressure measuring device can also record the pressure differences of the respective measuring chambers Δp_{P} and Δp_{R} to atmospheric pressure (index “P”, or “R” for potential or reference cell).

The measuring chambers are closed radially by tubular semipermeable, non-porous membranes, that can only be overcome diffusively by water. This exchange of water with the respective environment takes place until the difference in the chemical potentials of the water on both sides of the respective membrane disappears. The chemical potential and the activity of water are analytically linked in equilibrium. While the activity of the water outside is determined by the matrix potential Ψₘ prevailing there around the potential cell and the osmotic potential of the pore solution Ψ_{Π} or the reference potential Ψ₀, of the reference solution provided as a reference system around the reference cell, this is performed inside the two measuring chambers (index “MK”) by the osmotic potential Ψ_{MK}=Ψ_{P}=Ψ_{R} of the initially identical osmotic solutions.

Aqueous solutions are approximately incompressible. Therefore, the water exchange for an ideal, constant-volume measuring chamber of length L results in a changing osmotic pressure, which would occur in the chamber depending on the location of the external water potential. However, if the viscosity of the osmotic solution is sufficiently low, it equalizes the local pressure differences in the measuring chamber sufficiently quickly—an average pressure is established over the entire length of the measuring chamber. The pressure difference in the potential cell in relation to the atmospheric air pressure thus becomes:

$$Δ {p}_{P} = {\overline{Ψ}}_{w} - {Ψ}_{MK} , {\overline{Ψ}}_{w} = \frac{1}{L} {\int }_{0}^{L} {Ψ}_{w} ( x ) dx$$

Instead of a local water potential Ψ_{w} (x₀) at a location x₀ the pressure difference Δp_{P} is thus proportional to the soil water potential Ψ̅_{w} averaged over the length of the potential cell. Similarly, the pressure difference in the reference cell related to the atmospheric air pressure provides Δp_{R}=Ψ₀−Ψ_{MK} and thus creates a simultaneous reference to the reference potential Ψ₀, for example to Ψ₀=0 for standard conditions.

The pressure difference between the two measuring cells assumed to be ideal (constant volume) Δp_{P}−Δp_{R}=Ψ_{w}−Ψ₀ eliminates the osmotic potential prevailing in the measuring chambers with a good approximation. At the same time, this coupling of the two measuring cells causes a rotation of the measurement range, which means that the zero point of the pressure difference is defined exactly by the reference potential. If the reference potential is set for standard conditions, this means that the average water potential present in the test piece is determined by Ψ̅_{w}=Δp_{P}−Δp_{R}. If the pressure difference Δp=Δp_{P}−Δp_{R} between the two measuring chambers is determined directly by means of a pressure difference sensor, the average water potential according to Ψ̅_{w}=Δp already results directly from this measurement.

### Determination of Water and Matrix Potential

For example, the water potential can be measured for the reference potential set to standard conditions. Non-porous membrane materials for the measuring cells that are highly permeable to water such as for example PEBAX 1074, PBT/PEO block copolymers, silicone etc., are suitable for this purpose. In a large number of applications, particularly in humid climate conditions, this water potential also determines the matrix potential with sufficient accuracy, as the osmotic potential of the soil solution is often comparatively small (in terms of amount).

In arid climate conditions (dry, warm soils) concentrated salts (electrolytes) may be present in the soil solution as a result of the evaporation of the solvent, causing a non-negligible osmotic potential. In order to determine the matrix potential, the effect of this osmotic potential in the measured pressure difference has to be eliminated or reduced. If permeable polymer membranes are used to set up the measuring chambers for water, but not for the electrolyte ions, the osmotic potential can be compensated for approximately by a suitable shift in the reference potential. For this purpose, the reference system simply needs to be filled with a reference solution that is osmotically equivalent or similar to the soil solution. However, the electrolyte concentration in the soil solution varies as a function of precipitation, evaporation and salt inventory. Tracking the reference potential, for example controlled by conductivity measurements in the soil and the reference solution would be possible, but would involve increased measuring and control technical measures. Without such tracing, the matrix potential measured with a tensiometer set up in this way will therefore vary depending on the difference between the osmotic potentials of the electrolytes of the soil and reference solution.

The use of an ionomer membrane for constructing the measuring chambers would also allow ions to penetrate the membrane. The osmotic effects of the electrolytes on both sides of the potential cell then approximately cancel each other out. If the reference potential is defined by standard conditions, the matrix potential can now be determined exactly.

Potentially suitable ionomers are e.g. Nafion (DuPont), Flemion™ (Asahi Glas Co. Ltd) and DowMembrane® (Dow Chemical).

Nafion (“General Information on Nafion® membrane for Electrolysis”, Product Information, Bulletin 97-01, Rev. 04/2006), a sulfonated tetrafluoroethylene (PTFE) polymer, is highly permeable to water (and other polar substances) and cations, can be used at temperatures of up to 190° C. and is chemically extremely stable. Depending on its formulation, Nafion, which is only slightly stretchable, can, depending on its formulation, withstand extremely high mechanical stresses (see product information), which is significant with regard to the present application. If the pressure stability of Nafion membrane tubes up to 16 bar is taken into account, this results in a possibly broad measurement range for a tensiometer made with this material, within which the membrane tubes do not have to be mechanically supported against excess pressure that occurs.

For Nafion as a membrane material cations would preferably diffuse into the measuring chamber of the potential cell, followed by anions. This means that appropriately constructed measuring cells filled with a suitable osmotic solution can enable the desired equalization of the externally available electrolyte concentration, but with a time delay. During the concentration equalization however, there is an osmotic pressure difference which depends on the differences in the electrolyte concentrations on both sides of the membrane.

The preferred diffusion of cations through the Nation membrane on the other hand causes a charge separation. As a result of the accumulation of cations in the measuring chamber, a positive excess charge builds up here, accompanied by a negative excess charge in the external environment of the membrane. The resulting electrochemical potential difference Δφ causes the cations to migrate in the opposite direction to the concentration-driven diffusion. According to the Nernst-Planck equation, the resulting mass flux density \dot{n}ⱼ through the membrane can be described as the superposition of diffusion and migration \dot{n}ⱼ=−Dⱼ(∇cⱼ−F zⱼcⱼ/(RT)∇φ), where cⱼ is the concentration of cation j of charge zⱼ and Dⱼ is its diffusion coefficient in the membrane. F is the Faraday constant, R is the general gas constant and T the temperature. For negligibly small material flows (\dot{n}ⱼ→0 ∀ j), a dependency Δφ˜ΣΔcⱼ/(zⱼcⱼ) arises between the difference in the cation concentration and that of the electrochemical potential on both sides of the membrane to a first approximation.

The total number of ions outside and inside the measuring chamber is subject to the condition of charge neutrality. A positive excess charge built up in the measuring chamber therefore has to be equal in magnitude to the negative excess charge resulting outside. Both excess charges thus build up an electrical voltage U across the membrane that depends on the type and concentration of the electrolytes.

A suitable measurement of this voltage makes it possible on the one hand to prove that the electrolyte concentrations on both sides of the membranes are balanced, as the voltage must then disappear. However, by calibrating the voltage against the resulting osmotic pressure in the potential cell, it is also possible to compensate for the effect of fluctuating osmotic potential differences on the measurement of the matrix potential by reducing or eliminating the voltage-dependent pressure from the measured pressure difference Δp. In one embodiment, it is therefore provided that the tensiometer has a voltage measuring device for measuring an electric voltage between the inner side and outer side of the membrane of the potential cell.

In one embodiment of the method, it is provided accordingly that the matrix potential Ψ̅ₘ in the test piece is determined in measuring cells provided with ionomer membranes, by measuring the electric voltage U between the inner side and outer side of the membrane of the potential cell by means of the voltage measuring device, which after calibration makes it possible to determine a pressure that is caused by electrolyte concentration-dependent osmotic potential differences on both sides of the membrane and to eliminate this from the measured pressure difference Δp and thus determine the matrix potential Ψ̅ₘ.

### Compensation of the Pressure-Dependent Expansion of Non-Ideal Measuring Chambers

For non-ideal measuring chambers, the pressure-dependent elastic expansion of the measuring chambers can lead to a dilution of the osmotic solution, which essentially results from the pressure-dependent expansion of the membrane tubes. In the reversible range, this is proportional to the pressure difference between the inner and outer side of the respective membrane according to Hooke's law and can be taken into account within a reference-based measurement via a proportionality factor when determining the water or matrix potential. The influence of the elastic deformation can be taken into account in a first approximation by a constant α to be calibrated and the water or matrix potential can be calculated proportional to Δₚ (1+α). In one embodiment of the method, it is therefore provided that a pressure-dependent elastic deformation of the measuring chambers of the measuring cells is taken into account when determining the water and/or matrix potential

### Reference Point and Measurement Range

By arranging the reference cell in the reference chamber, a defined reference potential Ψ₀ can be created around which the measurement range can be adjusted according to the problem. As described in the introduction of the functional principle, this reference potential defines the zero point of the measurement range from which the measurement range rises with an adjustable width.

The composition of the reference solution and the pressure in the reference chamber determine this reference potential Ψ₀. By selecting a suitable reference system contributions from components in the water potential can be eliminated from the measurement result depending on the configuration. A reference solution in the reference chamber that is adapted osmotically to the measurement environment, for example, can be used to determine the matrix potential in isolation from the osmotic potential. Any pressure potential of interest can also be set in the reference chamber or imposed by the environment.

The measurement range can be determined according to the problem by filling the measuring chambers with a suitable osmotic solution.

In embodiments of the method it is therefore provided that the hygroscopic substance(s) and their concentrations within the osmotic solutions in the potential cell and in the reference cell and/or reference substance(s) and their concentrations in the reference solution are selected as a function of properties of the test piece and the type of membrane used.

In order to also be able to set a pressure in the reference chamber, it is provided in one embodiment that the reference chamber comprises a device for setting a pressure.

### Osmotic Solution

By dissolving suitable hygroscopic substances in defined concentrations in water, osmotic solutions with predeterminable osmotic potential can be produced. In addition to salts (electrolytes), water-soluble polymers such as polyethylene glycols, polyacrylamides, polysaccharides etc. are suitable for this purpose if they cannot penetrate the membrane (diffusively) or only at a negligible rate.

Polyethylene glycols (PEGs) are linearly linked polyethers, which are available in different chain lengths (repeating units of the base molecule [—CH2-CH2-O—]). They are biodegradable and are widely used in medicine and cosmetics. The solubility and the osmotic effectiveness of dissolved PEGs decreases with increasing chain length. Short chain PEGs, e.g. PEG 400 or PEG 800 (400 or 800 repeating units) can be mixed with water in almost any ratio, which means that different measurement ranges can be provided.

### Structural Variants of the Tensiometer

The reference system (in particular the reference chamber) can be sheathed for example by a suitable flexible plastic tube, e.g. made of polyurethane (PU), polyvinyl chloride (PVC) etc. If pressure stability of the reference system is required, thin-walled capillaries made from stainless steel or suitable pressure-stable plastics can be used. The tube/capillary ends are sealed from the outside or contain capillaries and valves for filling the reference chamber. In order to maintain the reference chamber at a defined pressure level, an external pressure (air pressure, hydrostatic water pressure) can applied via the wall of the reference chamber or supplied to the interior of the chamber via a capillary. The reference cell is arranged in a suitable manner in the reference chamber, for example by passing through it with radial symmetry.

The measuring cells may be identical to one another. In particular, the measuring cells then have the same lengths, diameters and wall thicknesses of the membranes and the non-porous, semipermeable membranes are similar in terms of their type and manufacture.

At the same temperature in the region of the tensiometer, the length of the reference cell or its volume can be reduced for example. The same applies to the entire reference system, which can then be combined with the now comparatively larger potential cell at a suitable position. In one embodiment it is therefore provided that the reference system is smaller than the potential cell and is positioned at a suitable location in the tensiometer.

In one embodiment it is provided that the tensiometer has an element that extends along a longitudinal axis of the measuring chambers of the measuring cells, which is placed in the potential cell or in each of the potential and reference cells and is arranged centrally in relation to a cross-section of the measuring chambers of the measuring cells perpendicular to the longitudinal axis. The element can be configured to be cylindrical for example (round rod) or also with a different cross-section as a profiled rod. In this way the volume of the measuring chambers in which the osmotic solutions are located is reduced. Taking into account the unchanged geometry of the enveloping membrane, this reduction in volume leads to a faster adjustment of the pressures in the measuring chambers, i.e. the reaction time of the tensiometer can be shortened in this way. The rod-shaped element has a cross-section that means it can be integrated into the respective measuring chamber such that a volume fillable with the osmotic solution and distributed as evenly as possible radially around the element remains between the element and inner membrane surface. A radially symmetrical integration of the element into the respective measuring chamber is optimal. The element is matched to the length and diameter of the respective measuring chamber such that a minimum reaction time of the tensiometer is achieved. The element is configured to be flexible or rigid and can have webs that are arranged in a spiral, straight line or ring on its surface to define its position and are in linear or point-like contact with the membrane wall. Alternatively, a fleece or net-like stocking made from a suitable (incompressible) plastic can be used to define the position.

If a volume-reducing element is integrated into the potential chamber as described above, this enables a further design of the tensiometer in which the reference system is integrated into the measuring chamber of the potential cell. In one embodiment it is therefore provided that the reference system is integrated into the potential cell over its length. In a further embodiment it is provided that the reference system at least partly forms the element according to the embodiment described above. Equal lengths of both measuring cells then still enable measurement over areas with locally varying temperatures. The hollow cylindrical space remaining in the measuring chamber of the potential cell due to the integration of the reference system causes the desired reduction of the volume filled with the osmotic solution and thus the reduction of the reaction time of the tensiometer. Chambers, membranes and the wall of the reference system can be manufactured such that the volumes of the potential and reference cells filled with the osmotic solution match or that a reproducible geometric relationship between the wall thicknesses of the membranes, membrane surfaces and volumes of the osmotic solutions ensures that the osmotic pressure is set more quickly or equally quickly in the reference cell compared to the potential cell, regardless of the (possibly different) membrane materials used.

### Mechanical Stabilization

The measuring chambers can be integrated into capillary tubes or, in particular tubular braids, which are provided with water-permeable recesses and into which the respective measuring cells are fitted/pressed flush. In one embodiment, it is therefore provided that the tensiometer has capillary tubes provided with permeable cavities and/or pores, into which the membranes of the measuring cells are each fitted flush.

Alternatively, the membranes of the measuring cells can be at least partly integrated as a filling into the recesses of the capillary tubes or braids. Therefore, in a further embodiment it is provided that the tensiometer is provided with capillary tubes provided with cavities and/or pores, or in particular has tubular braids, wherein the membranes of the measuring cells are at least partially integrated into the cavities.

Both variants can reduce the elastic expansion of the measuring cells so that the volumes in the measuring chambers can be kept approximately constant even at high pressures. This type of stabilization also enables a reduction in the wall thickness of the membranes and the use of different polymers or ionomers with approximately constant expansion behavior. In addition, the membranes can be protected from external mechanical influences.

For example, the capillary tubes can have slotted recesses, be made of porous sintered metals or ceramics or also be made of plastic. Braids can consist of metallic and/or non-metallic fibers or comprise the latter and envelop and/or embed the membrane with a defined mesh size.

### Integrated Temperature Measurement

The tensiometer can be equipped with temperature sensors that are integrated into the potential cell, the reference cell or both measuring cells, depending on their design. In one embodiment, it is therefore provided that the tensiometer has at least one temperature sensor in or on the potential cell and/or in or on the reference cell. In particular, the at least one temperature sensor allows at least the temperature in the potential cell or the reference cell to be recorded. This allows the temperature to be provided as an important parameter of the ambient conditions. Measuring the temperature difference between the measuring cells also makes it possible to compensate for temperature-dependent differential pressure dependencies between the measuring cells or to specify corresponding uncertainties in the measurement result. The calibration of temperature-dependent dependencies of the pressure difference pressure Δp is preferably carried out experimentally as a function of specified temperatures and temperature differences.

The temperature sensor can for example be integrated as a wire-shaped resistance sensor along the longitudinal axis of the measuring cell.

The elements used for volume reduction can accommodate or form at least part of these temperature sensors. Therefore, in a further embodiment it is provided that the element forms at least part of the temperature sensor.

### Integrated Voltage Measurement

The tensiometer can be provided with a measuring arrangement, comprising a voltage measuring device with corresponding contact, which records the electrical potential difference between the measuring chamber of the potential cell and its immediate environment as the electrical voltage. For a measuring cell constructed with an ionomer membrane, it is then possible to reduce or eliminate interference when measuring the matrix potential. This voltage can be stored on the tensiometer board as a function of the osmotic potential difference between two sides of the membrane caused by salts, e.g. calibrated as a pressure function, and used to reduce or eliminate varying osmotic potential differences superimposed on the matrix potential. For calibration, the electrical voltage between the inner side of the membrane (in the measuring chamber) and its outside (near the membrane surface) is preferably determined experimentally as a function of specified ion concentrations and the associated osmotic pressure in the potential cell is determined.

The element used for volume reduction within the measuring chamber of the potential cell can form one of the electrical contacts for measuring the voltage. Therefore, in a further embodiment it is provided that the element forms at least part of an electrical contact to the osmotic solution in the potential cell, which is connected to the voltage measuring device. For this purpose, the surface of the element is at least partially electrically conductive.

The capillary tube or braid arranged around the potential cell can be (at least partially) electrically conductive and form at least part of the external electrical contact for the voltage measurement. Therefore, in one embodiment, it is provided that at least the capillary tube or braid in which the potential cell is arranged is at least partially electrically conductive and forms an electrical contact to the (moist) outer membrane surface to which the voltage measuring device is connected. For this purpose, the braid can comprise metallic fibers for example through which the external potential can be tapped.

### Filling the Measuring Chambers

The measuring chambers can each have inlet and outlet valves at both ends. Therefore, in one embodiment, the measuring cells are each provided with an inlet/outlet valve at both ends. This allows the measuring chambers or measuring cells to be filled, emptied, rinsed and cleaned. In particular, this makes it possible to replace the osmotic solution in a tubular tensiometer installed in the test piece, e.g. in the floor, without removing it and thus without disturbing the measuring environment in the course of this maintenance work on the tensiometer. In this context, a pumping device can be installed and/or used temporarily or permanently to empty or fill the measuring cells or to equalize differences in the concentration of the osmotic solutions between the measuring cells.

For adjustment, correction and status testing, the concentrations or corresponding concentration differences between the measuring cells can be recorded temporarily or permanently using a suitable measuring device. When using hygroscopic salts, for example, the conductivity can be measured to assess the concentration. In one embodiment, the tensiometer therefore has a concentration measuring device that is set up to detect at least one concentration difference of a hygroscopic substance within the osmotic solutions in the measuring cells.

The hygroscopic substance(s) in the measuring chambers and/or the reference substance(s) can be selected or preset depending on properties of the test piece and the type of membrane used. This allows the position and size of the measurement range to be specified and adjusted during operation. The measurement range can be adjusted for example, by the concentration(s) and type(s) of the hygroscopic substance(s), as this allows the osmotic potential of the solutions to be specifically influenced.

Furthermore, the reference solution can be used in a composition that is osmotically equivalent to the test piece. If a membrane impermeable to ions is used as the membrane material, the osmotic potential can be eliminated by measurement, whereby the pressure difference recorded between the measuring chambers approximately determines the matrix potential. In one embodiment of the method, the osmotic reference solution used is therefore an aqueous solution with an osmotic composition approximately equivalent to that of the test piece.

### Data Processing

The pressure value determined at the pressure sensor is prepared by a suitable electronic circuit for data processing as a suitable signal, for example as a data value or data packet, converted into the desired representation form for the water or matrix potential, suitably corrected, and if necessary stored together with other parameters (temperatures, system parameters), output, displayed and/or made available via a data bus-capable output. The electronic components and program codes required for this are implemented in a tensiometer board.

### Installation-Dependent Variants

Due to the linear geometries of the potential cell and reference system, which can be flexibly configured, such a tensiometer can be guided as a common strand through the soil body, substrate or medium.

If the tensiometer is to be inserted into the test piece, the reference system can be mechanically reinforced on the outside, e.g. with a perforated hollow profile. The same applies in this case to the potential cell exposed to the test piece.

Due to its linear, flexible or rigid design, the type and length of the tensiometer can be adapted to a specific problem, e.g. to the heterogeneity of the site, the size of the pedon, the root density distribution, etc., as well as to the type of installation into test piece, e.g. by laying it in suitably prepared areas of the test piece, e.g. accessible through slots or pre-drilling, or by piercing it (again after pre-drilling if necessary to avoid local material compaction).

### FIELD OF APPLICATION

In addition to being used in soils, the tensiometer can also be used in other substrates or media to determine the existing water status, for example to precisely determine the relative humidity close to 100%, i.e. where conventional humidity sensors are generally inaccurate. Maintaining the highest possible humidity while avoiding the formation of condensation is necessary for the storage of fruit for example.

### SUMMARY

The invention relates to a linear water potential sensor (tensiometer) of selectable length with adjustable measurement range for use in dry and moist, heterogeneously composed soils, substrates or media and a reference-based method for the representative determination of the water potential, e.g. along a horizontal in this test piece by spatially averaging the locally present water potential along the tensiometer.

The tensiometer and the described method enable the determination of the water or matrix potential averaged over the test piece, in particular a pedon-scale soil body. Furthermore, a suitable selection of the hygroscopic substance(s) and their concentration in the used osmotic solution and/or the reference substance(s) and their concentration in the used reference solution can be used to set a measurement range in position and size which is optimally adapted to the dynamic range of the water potential in the test piece.

In this way, a diffusion-solid tensiometer referenced via a definable internal standard is created, which can be adapted to the respective measurement problem in terms of its spatial averaging behavior and the size and position of the measurement range.

### BRIEF DESCRIPTION OF THE DRAWING FIGURES

In the following the invention is explained in more detail with reference to the Figures using preferred embodiments. In the Figures:

FIG. 1 shows a schematic representation of an embodiment of the tensiometer;

FIG. 2 shows a schematic representation of a section of the tensiometer for illustrating a further embodiment of the tensiometer;

FIG. 3 shows a schematic representation of a section of the tensiometer for clarifying a further embodiment of the tensiometer;

FIG. 4 shows a schematic representation of a further embodiment of the tensiometer;

FIG. 5 shows a schematic flowchart of an embodiment of the method for determining a spatially averaged water potential in a test piece.

### DETAILED DESCRIPTION OF THE INVENTION

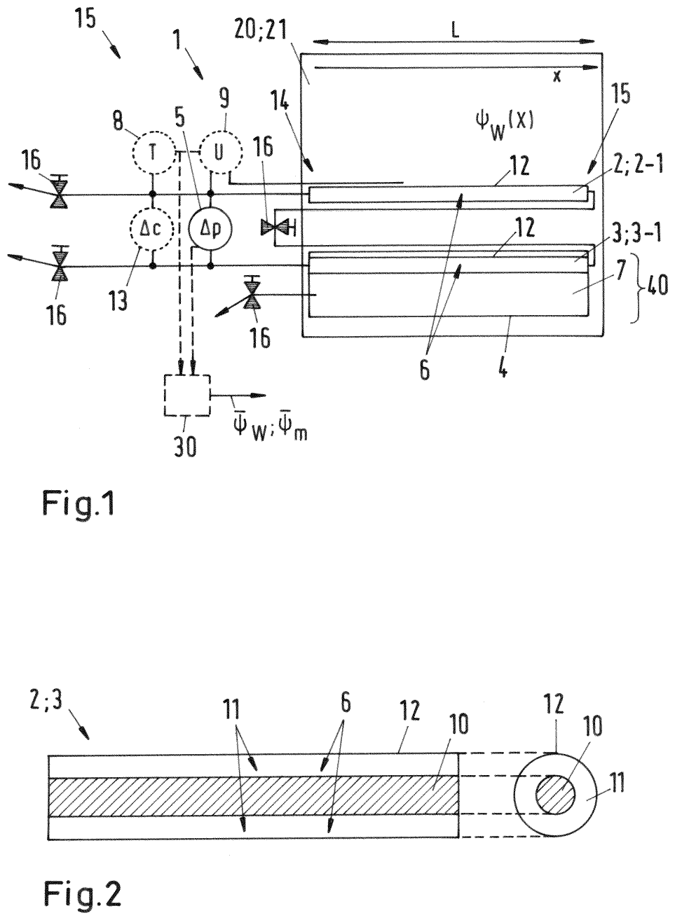

FIG. 1 shows a schematic representation of an embodiment of the tensiometer 1. The tensiometer 1 comprises a potential cell 2 and a reference system 40 with a reference cell 3 and with a reference chamber 4 and a pressure measuring device 5.

The potential cell 2 comprises a closable measuring chamber 2-1 sheathed in a tubular semipermeable membrane 12. The membrane 12 is a water-permeable polymer (for example silicone) or an ionomer (for example Nafion) or a material disclosed in the general description or a functionally equivalent material.

The reference cell 3 comprises a closable measuring chamber 3-1 sheathed in a tubular semipermeable membrane 12. The reference cell 3 is in particular identical in structure to the potential cell 2. The diameter, length and wall thickness of the membranes 12 of both measuring chambers 2-1, 3-1 as well as the membrane material are identical. The reference cell 3 is integrated into the reference chamber 4.

Alternatively, it can also be provided in principle, that the reference system 40 is configured to be smaller than the potential cell 2 and positioned at a suitable location in the tensiometer 1.

The potential cell 2 and the reference cell 3 are filled with an osmotic solution 6, wherein the solution 6 is the same in both measuring chambers 2-1, 3-1. The osmotic solution 6 can be prepared with salts and/or water-soluble polymers, e.g. PEGs, with a suitable chain length.

The reference cell 3 integrated into the reference chamber 4 is surrounded by the reference solution 7. This reference solution 7 together with the reference potential defines the reference point for measuring the water potential. The reference solution 7 can be water for example. The reference chamber 4 is formed for example by a liquid-tight tube or bag made of a suitable plastic, such as polyurethane (PU) or polyvinyl chloride (PVC) etc.

The pressure measuring device 5 makes it possible to detect at least the pressure difference Δp between the potential cell 2 and the reference cell 3, for example by means of a pressure difference sensor.

The potential cell 2 and the reference system 40 (reference cell 3 and reference chamber 4) are exposed to the same environmental conditions. In particular, the temperature should be the same in the measuring cells 2, 3. At least one temperature sensor 8 can be provided for measuring a temperature T by which at least one temperature of the potential cell 2 is determined.

The potential cell 2 and the reference system 40 (reference cell 3 and reference chamber 4) are arranged in a test piece 20, in particular in soil 21, in which the water potential or the matrix potential is to be determined. In particular, it is provided that the measuring chambers 2, 3 are guided through the test piece 20, 21 closely adjacent to one another, for example as a strand. This is shown schematically in FIG. 1 in simplified form, where the measuring cells 2, 3 and the reference chamber 4 extend in a symbolic direction x in the test piece 20. When using the tensiometer 1 in the soil the measuring cells 2, 3 and the reference chamber 4 can extend in any spatial direction, so that a position of the tensiometer 1 in the test piece 20 can be determined according to the respective question and thus the range over which the averaged water or matrix potential is to be recorded. The measuring cells 2, 3 and the reference chamber 4 can have a length L within a range of a few centimeters to several meters.

The tensiometer 1 can be used to determine the water or matrix potential Ψ̅_{w/m} in the test piece 20, wherein the pressure difference Δp between the potential cell 2 and the reference cell 3 is measured by means of the pressure measuring device 5. On the basis of this pressure difference Δp the water or matrix potential in the test piece 20—soil 21 or another substrate—is measured and provided. This is performed in particular, after equilibriums of the water activities between the osmotic solutions 6 in the measuring cells 2, 3 on the one hand and the waters in their environment (test piece 20, reference chamber 4) on the other hand have been established.

To determine the water or matrix potential the tensiometer 1 has a tensiometer board 30, which comprises electronic components, for example a programmed microcontroller and a memory. The tensiometer board 30 is in particular part of the tensiometer 1. In particular, the tensiometer board 30 evaluates the signal from the pressure measuring device 5 and uses this to generate a measurement value for the water or matrix potential Ψ̅_{w/m} averaged over length L.

It can be provided that the tensiometer 1 has a voltage measuring device 9 that detects an electrical voltage U between an inner side and an outer side of the potential cell 2. It is then provided that the voltage U is determined by means of the voltage measuring device 9 to correct electrolyte concentration-dependent osmotic potential differences between the potential cell 2 and an external space and based on a calibration of the associating pressure in the potential cell 2 is reduced or eliminated from the determined pressure difference Δp in order to calculate the matrix potential Ψ̅m averaged over length L for the water state in the test piece 20, in particular in the soil 21.

It can be provided that the tensiometer 1 has a rod-shaped element 10 inserted into the potential cell 2 and the reference cell 3 respectively, which extends along the measuring chambers 2-1 and 3-1 is arranged centrally therein. This embodiment of the tensiometer 1 is illustrated schematically as an enlarged section in FIG. 2. The element 10 can be configured as a profile or round rod for example and is used for reducing the volume of the measuring chamber in which the osmotic solution is located. In the embodiment shown, the measuring chamber volume is reduced to a hollow cylindrical annular space 11 between the element 10 and the inner surface of the membrane 12. For this purpose, the element 10 is arranged centrally in relation to the cross-section of the respective measuring cells 2 and 3. For this purpose, positioning means can also be used which maintain the central arrangement of the element 10, as disclosed in the general description. By reducing the volume of the measuring chamber, the setting time for the pressure within the measuring cells 2, 3 is reduced in particular, so that the water potential can be determined with greater resolution. The element 10 is particularly flexible so that the flexibility of the tubular measuring cells 2, 3 is not restricted.

Furthermore, it can be provided that the element 10 comprises at least a part of a temperature sensor 8 and/or an electrical contact to the osmotic solution for the voltage measuring device 9. For this purpose, a temperature-dependent resistor is arranged for example in the element 10, for example in the profiled or round rod, and/or the profiled or round rod externally forms at least partly an electrically conductive electrode, which is in contact with a voltage measuring device 9.

It can also be provided that the reference system 40 is integrated into the potential cell 2 over its length. In particular, it can then be provided that the reference system 40 at least partially forms the element 10.

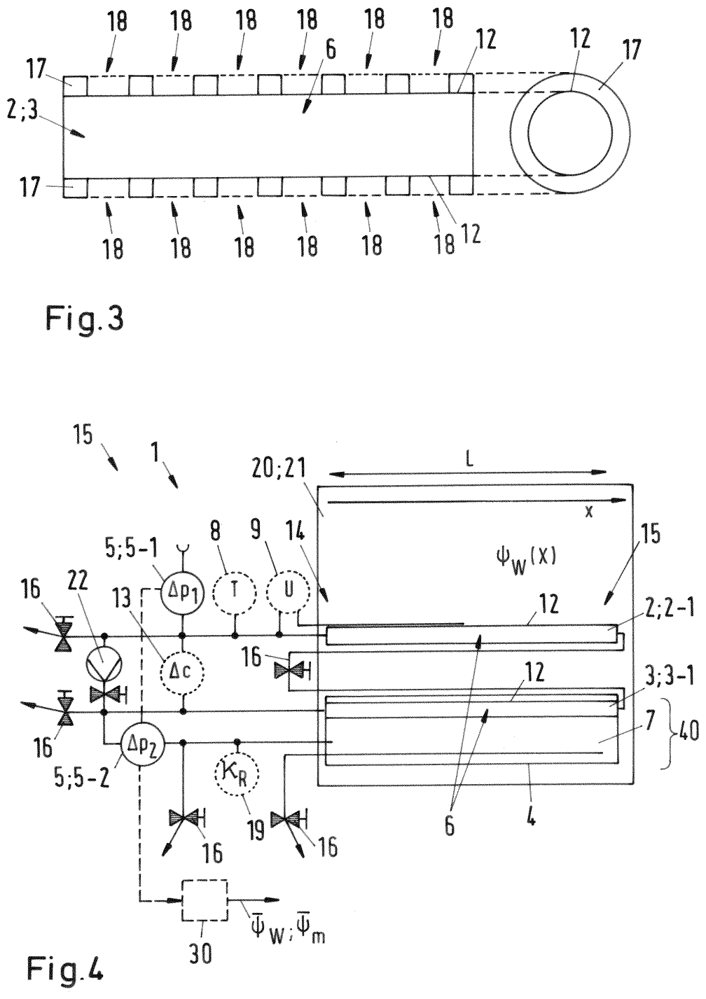

The FIG. 3 shows a schematic representation of a section of a further embodiment of the tensiometer, in which it is provided that the tensiometer has capillary tubes 17 with water-permeable perforated, slotted or porous cavities 18. Instead of a rigid capillary tube 17, braids, in particular tubular braids, can also be provided. These braids can consist of or comprise metallic and/or non-metallic fibers. The membranes 12 of the measuring cells 2 or 3 are either integrated flush into the capillary tubes 17, fill the cavities in the wall of the capillary tubes 17 or are flush/force-fit sheathed by and/or embedded in a braid with a defined mesh width. The water in the respective environment is in contact with the membranes 12 of the measuring cells 2 and 3 via the cavities 18. This provides protection against mechanical influences and limits the varying elastic explanation of the membranes 12 caused by the changing pressure in the measuring cells 2, 3. The capillary tubes/braids 17 can be made of plastic, metal or a ceramic. They can also consist of the material of a suitable semipermeable membrane that is permeable to water.

Furthermore, it can be provided that the pressure-dependent elastic deformation of the measuring cells 2, 3 is calibrated in the manner described above and taken into account when determining the water or matrix potential.

Furthermore, it can be provided that at least the capillary tube/braid 17, in which the potential cell 2 is arranged, is at least partially electrically conductive and is configured to be at least partly electrically conductive and forms an electric contact to the (moist) outer membrane surface, to which the voltage measuring device 9 is connected. For example, for this purpose the braid can comprise one or more metallic fibers by which an external potential can be tapped.

It can be provided that the tensiometer 1 has a concentration measuring device 13 (FIG. 1), which detects the concentration difference Δc of the hygroscopic substance between the measuring cells 2, 3 using the tensiometer board 30. This makes it possible to check whether sufficiently similar concentrations are present in the measuring cells 2, 3 after filling or changing the osmotic solution 6.

It can be provided that the measuring cells 2 and 3 each have inlet and outlet valves 16 at both ends 14, 15 (FIG. 1). This can facilitate the filling and emptying of the measuring cells 2, 3. It can also be provided that the reference chamber 4 has an inlet and outlet valve 16. In the embodiment shown in FIG. 1, the measuring cells 2 and 3 can be connected to one another via the inlet and outlet valve 16. This enables the concentration of the osmotic solutions 6 to be equalized between the measuring cells 2, 3 and makes it easier to fill them evenly.

FIG. 4 shows a schematic representation of a further embodiment of the tensiometer 1. The embodiment is configured in principle like the embodiments shown in FIG. 1, the same reference signs denote the same features and terms. In this embodiment it is provided that the pressure measuring device 5 has a first pressure difference sensor 5-1 and a second pressure difference sensor 5-2. The first pressure difference sensor 5-1 detects the pressure difference Δp₁ between the potential cell 2 and the atmosphere. The second pressure difference sensor 5-2 detects a pressure difference Δp₂ between the reference cell 3 and the atmosphere. From the difference between the two pressure differences Δp₁, Δp₂ the pressure difference Δp between the two measuring cells 2, 3 can be determined, from which the water potential Pw or matrix potential I'm averaged over the length L can be determined in the manner described above.

Furthermore, it can be provided that the tensiometer 1 has a pump device 22. This can be installed and/or used temporarily to equalize differences in concentration between two measuring chambers 2-1, 3-1, for example, after an exchange of the osmotic solution 6 in the measuring chambers 2-1, 3-1.

Furthermore, it can be provided that the tensiometer 1 has a conductivity sensor 19 for determining the conductivity KR of the reference solution 7. This makes it possible to check and set an ionic strength or concentration of the reference solution 7 in the reference chamber 4.

In a preparatory step, it may be provided that the hygroscopic substance(s) in the measuring chambers and/or the reference substance(s) in the reference chamber 4 are selected as a function of properties of the test piece. The same applies to the concentrations of the respective osmotic solutions. The selection of the hygroscopic substance(s) and/or the reference substance(s) can be based on empirical findings obtained from set series. For example, it can be provided that by means of suitable reference substance(s) an aqueous solution with an osmotically equivalent composition to the test piece can be prepared. In particular, this allows the matrix potential to be determined directly via the recorded pressure difference if a suitable polymer is used as the membrane.

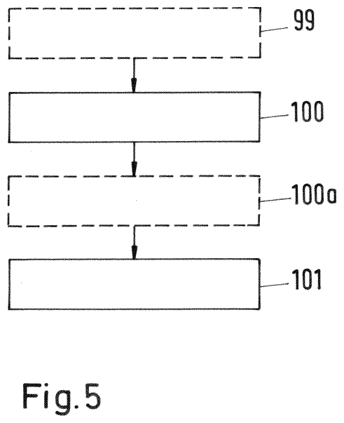

FIG. 5 shows a schematic flowchart of an embodiment of the method for determining a spatially averaged water potential in a test piece.

In one measure 100 at least one pressure difference between the measuring chambers of the potential cell and the reference cell is detected by means of a tensiometer according to one of the embodiments described above.

In one measure 101 based on the determined pressure difference as a function of the semipermeable membrane used and the reference potential the water and/or matrix potential in the test piece, averaged over the length of the potential cell is determined and provided as a measurement value. This is performed in particular by means of the tensiometer board.

It can be provided in one measure 100a that the matrix potential in the test piece is determined in measuring cells provided with ionomer membranes, by detecting the electrical voltage between the inner side and the outer side of the membrane of the potential cell by means of the voltage measuring device, which after calibration enables a pressure to be determined which is caused by electrolyte concentration-dependent osmotic potential differences on both sides of the membrane, and to eliminate this from the measured pressure difference and thus determine the matrix potential. This is also performed using the tensiometer board. The determined matrix potential is also provided.

In a preparatory measure 99 it can be provided that the hygroscopic substance(s) and their concentrations within the osmotic solutions in the potential cell and in the reference cell as well as their concentrations in the reference solution are selected as a function of properties of the test piece and the type of membrane used. The selection of hygroscopic substance(s) can be based for example on empirical findings from the test series.

For example, it can be provided in measure 99 that an aqueous solution with a composition that is approximately osmotically equivalent to the test piece is used as the reference solution. In particular, this allows the matrix potential to be determined directly via the recorded pressure difference since the osmotic potential and the reference potential specified by the reference substance have the same value.

It can be provided in measure 101 that a pressure-dependent elastic deformation of the measuring chambers of the measuring cells is taken into account when determining the water and/or matrix potential.

The tensiometer described in this disclosure and the method described enable in particular the determination of a spatially averaged water potential over a test piece. Furthermore, the matrix potential can also be determined by means of the embodiments described. In addition, a measurement range can be set via a suitable selection of the osmotic solution and/or the reference solution, so that the tensiometer can be adapted to properties of the test piece.

### LIST OF REFERENCE SIGNS

1 tensiometer
2 potential cell
2-1 measuring chamber
3 reference cell
3-1 measuring chamber
4 reference chamber
5 pressure measuring device
5-1 pressure difference sensor
5-2 pressure difference sensor
6 osmotic solution
7 osmotic reference solution
8 temperature sensor
9 voltage measuring device
10 element
11 hollow-cylindrical annular space
12 membrane
13 concentration measuring device
14 end
15 end
16 inlet/outlet valve
17 capillary tube (or braid)
18 cavity
19 conductivity sensor
20 test piece
21 soil
22 pump device
30 tensiometer board
L length of the measuring chambers
T temperature
U electric voltage
X installation direction of the tensiometer
Δ_{c} concentration difference
κ_{R} conductivity
Δₚ pressure difference
Δp₁ pressure difference
Δp₂ pressure difference
Ψ̅ₘ averaged matrix potential
Ψ̅_{w} averaged water potential

What is claimed is:

1. A tensiometer, comprising:
a measuring cell of selectable length, referred to as a potential cell, which comprises a closable measuring chamber sheathed in a tubular semi-permeable membrane and can be installed in a test piece;
a reference system comprising a reference chamber and a measuring cell of selectable length, referred to as a reference cell, which comprises a closable measuring chamber sheathed in a tubular semipermeable membrane, wherein the reference cell is integrated into the reference chamber of the reference system, and wherein the reference chamber contains an osmotic reference solution or can be filled with such a solution, wherein the potential cell and the reference cell are filled or can be filled with an osmotic solution; and
a pressure measuring device, which is configured to measure at least one pressure difference between the measuring chambers of the potential cell and the reference cell.

2. The tensiometer according to claim 1, wherein the reference cell has an identical structure to the potential cell.

3. The tensiometer according to claim 1, characterized in that the reference system is smaller than the potential cell and is positioned at a suitable location in the tensiometer.

4. The tensiometer according to claim 1, wherein the reference chamber comprises a device for setting a pressure.

5. The tensiometer according to claim 1, including a voltage measuring device for measuring an electrical voltage between the inner side and the outer side of the membrane of the potential cell.

6. The tensiometer according to claim 1, wherein an element which is introduced into the potential cell or into each of the potential and reference cells, extends along a longitudinal axis of the measuring chambers of the measuring cells and is arranged centrally with respect to a cross-section of the measuring chambers of the measuring cells oriented perpendicular to the longitudinal axis.

7. The tensiometer according to claim 1, wherein the reference system is integrated into the potential cell over its length.

8. The tensiometer according to claim 7, wherein an element which is introduced into the potential cell or into each of the potential and reference cells, extends along a longitudinal axis of the measuring chambers of the measuring cells and is arranged centrally with respect to a cross-section of the measuring chambers of the measuring cells oriented perpendicular to the longitudinal axis, and wherein the reference system at least partly forms the element.

9. The tensiometer according to claim 1, comprising capillary tubes or braids provided with permeable cavities and/or pores into which the membranes of the measuring cells that are fitted flush respectively or are at least partially integrated into the cavities.

10. The tensiometer according to claim 1, including at least one temperature sensor in or on the potential cell and/or in or on the reference cell.

11. The tensiometer according to claim 10, wherein an element is introduced into the potential cell or into each of the potential and reference cells, and forms at least a part of the temperature sensor and/or of an electrical contact to the osmotic solution in the potential cell, which is connected to the voltage measuring device.

12. The tensiometer according to claim 9, wherein at least the capillary tube or the braid, in which the potential cell is arranged, is at least partially electrically conductive and forms an electrical contact to the (moist) outer membrane surface, to which the voltage measuring device is connected.

13. The tensiometer according to claim 1, wherein the measuring cells each have an inlet/outlet valve at both ends.

14. The tensiometer according to claim 1 including a concentration measuring device, which is configured to detect at least one concentration difference of a hygroscopic substance within the osmotic solutions in the measuring cells.

15. A method for determining a spatially averaged water potential (Ψ̅_{w}) in a test piece, wherein at least the pressure difference between the measuring chambers of the potential cell and the reference cell is detected by means of the tensiometer according to claim 1, which due to its arrangement inside the reference chamber of the reference system permits the reference to a selectable reference potential, wherein the water and/or matrix potential (Ψ̅_{w/m}) in the test piece, averaged over the length of the potential cell, is determined on the basis of the detected pressure difference (Δp) according to the semi-permeable membrane used and the reference potential and is provided as a measured value.

16. The method according to claim 15, wherein the matrix potential (Ψ̅ₘ) in the test piece is determined in measuring cells provided with ionomer membranes by detecting the electrical voltage between the inner side and the outer side of the membrane of the potential cell by means of the voltage measuring device, which after calibration makes it possible to determine a pressure which is created by electrolyte concentration-dependent osmotic potential differences on both sides of the membrane, and to eliminate this from the measured pressure difference and thus determine the matrix potential (Ψ̅ₘ).

17. The method according to claim 15, wherein the osmotic solutions of the respective potential cell and the reference cell include a hygroscopic substance(s), wherein the osmotic reference solution comprises a reference substance(s), and wherein the hygroscopic substance(s) and their concentrations within the osmotic solutions in the potential cell and in the reference cell and/or reference substance(s) and their concentrations in the reference solution are selected as a function of properties of the test piece and the type of membrane used.

18. The method according to claim 15, wherein an aqueous solution with an osmotic composition almost equivalent to that of the test piece is used as the osmotic reference solution.

19. The method according to claim 15, wherein a pressure-dependent elastic deformation of the measuring chambers of the measuring cells is taken into account when determining the water and/or matrix potential (Ψ̅_{w/m}).

20. The tensiometer according to claim 6, including at least one temperature sensor in or on the potential cell and/or in or on the reference cell.
